(19) United States
(12) Patent Application Publication
(10) Pub. No.: US 2023/0172551 A1
(43) Pub. Date: Jun. 8, 2023

(54) METHOD AND DEVICE FOR PROVIDING PERSONALISED HAEMODIALYSIS FOR A SUBJECT

(71) Applicant: RENALYX HEALTH SYSTEMS PRIVATE LIMITED, Bangalore (IN)

(72) Inventors: Mahabaleswara Rama BHATT, Bangalore (IN); Ajay SHARMA, Bangalore (IN); Shyam Vasudeva RAO, Bangalore (IN); Vincent LLYOD, Bangalore (IN)

(21) Appl. No.: 17/917,626

(22) PCT Filed: Apr. 9, 2021
(86) PCT No.: PCT/IB2021/052952

§ 371 Date: Oct. 7, 2022

(30) Foreign Application Priority Data
Apr. 9, 2020 (IN) 201941040826

(51) Int. Cl. A61B 5/00; A61B 5/11; A61B 5/145; A61M 1/16

(52) U.S. Cl. CPC A61B 5/6866; A61B 5/0075; A61B 5/1102; A61B 5/14546; A61M 1/1607; A61M 2205/18

(57) ABSTRACT

The present disclosure is related to a method and system for providing personalised haemodialysis for subject. The method includes obtaining concentration of electrolytes and of metabolic content in blood sample flowing into and out of dialyser through first blood bypass tube and second blood bypass tube, respectively. The first and the second blood bypass tube are arranged in first sensor and second sensor. Similarly, concentration of electrolytes and metabolic content in dialysate fluid flowing into and out of dialyser through first and second dialysate tube, respectively. The first dialysate tube and second dialysate tube are arranged to pass through third sensor and fourth sensor. Further, variations are identified in concentration obtained for electrolytes and metabolic content in blood sample with respect to concentration obtained for electrolytes and metabolic content in dialysate fluid, respectively. Thereafter, removal of electrolytes and metabolic content is performed from blood sample.

### TECHNICAL FIELD

The present disclosure generally relates to field of haemodialysis. Particularly, but not exclusively, the present disclosure relates to a method and a system for providing personalised haemodialysis for a subject.

### BACKGROUND

Haemodialysis is a process of purifying blood in three aspects, namely, undesirable molecules such as urea, uremic toxins, creatinine, and the like produced by human body, excess fluid accumulated due to lack of excretion through kidney and concentration of different electrolytes in the blood which may imbalanced and requires rebalancing to avoid several dysfunction of human tissue. During intradialysis, monitoring of multiple metabolic waste products such as, urea, creatinine, carbohydrates/triglycerides, sodium bicarbonates, uremic toxins, albumin, haemoglobin, and haematocrit are very crucial due to their imbalances which are inevitable, while dialysis is in progress. For example, serum creatinine level is known to be a good biomarker for an estimation of glomerular filtration. There are many reasons for creatinine and urea levels to increase in the blood and mainly due to renal failure or decreased kidney function.

Generally, variations in concentration of multiple electrolytes occur in human body due to both diffusion and convective processes between blood and dialysis fluid paths which may ultimately harm a patient under dialysis. The dialysis fluid is a composition of mixture of electrolytes that are also present in the blood (for example, Na+, K+, Cl−, Ca++, Mg++, bicarbonate etc). During the progression of dialysis, due to exchange between blood and dialysis fluid, the concentration may change in both sides. Furthermore, mixture can be varied depending on metabolic condition of the patient. The imbalance in electrolyte may influence Electrocardiogram (ECG) which may reflect changes in states to reflect like hyperkalaemia or hypokalaemia, hypercalcemia, hypocalcaemia, etc.

In existing dialysis systems, though the patient is a part of the bloodline circuit of dialysis process, neither blood electrolytes variations nor its dependent parameters are utilized for monitoring, controlling and alarming system on any eventualities. Typically, dialysis control restricts to physical parameters like flow/pressure in both blood and dialysis path.

Further, there is a large gap in conventional HD where onetime generic prescription is provided with fixed dosage, when compared to natural human kidney, which is capable of maintaining complete homeostasis using complex sensing and biofeedback system to respond for any changing condition. Typically, the conventional HD setup exploits convection or diffusion by three factors. Firstly, conventional setups sense only extracorporeal hydraulic circuit parameters using pressure sensors or flow sensors at the arterial and venous part and some allied sensors like, optical or ultrasonic sensor to monitor situation caused by air bubbles and haemolytic conditions of the blood. Secondly, the conventional setups sense physical parameters such as, pressure sensors/flow sensors on both sides of hemodialyzer to monitor transmembrane pressure, and thirdly by sensing conductivity on inlet and outlet sides of dialysis fluid circuit to monitor dialysate composition in an indirect way in the dialysis fluid hydraulic circuit apart from blood leak sensing. The above process is managed based on prescription suggested by nephrologists in a generic way decided during diagnosis.

Further, Dry weight (DW) is termed as normal weight without any extra fluid in the body. When there is a kidney failure, the body depends on dialysis to get rid of the extra fluid and wastes that build up in body during treatments. Dry weight (DW) is an important clinical target in haemodialysis, which is influenced by many factors and may change in response to multiple variables, including presence of edema, muscle cramps, or changes in blood pressure. Further, changes in DW are influenced by cardiothoracic rate (CTR), natriuretic peptide levels, and intracardiac pressure or diameter of the inferior vena cava obtained via echocardiogram. Hence, it is very much essential to monitor and eventual auto alarm for stopping the haemodialysis process.

Currently in intra dialysis process, continuous removal of fluid from individual dialysis patient may lead to either myocardial infarctions or any other cardiac related fatal issues, which demands to have the balance in either electrolytes parameters or physical parameters in an extracorporeal blood arm of the dialysis. Additionally, this gives rise to problem of dry weight maintenance and requires an immediate alert system.

### SUMMARY

In an embodiment, the present disclosure may relate to a method for providing personalised haemodialysis for a subject. The method includes obtaining concentration of one or more electrolytes and of metabolic content in a blood sample of a subject flowing into a dialyser and out of the dialyser through a first blood bypass tube and a second blood bypass tube, respectively. The first blood bypass tube and the second blood bypass tube are arranged in a first sensor and a second sensor configured in the haemodialysis filtering device, respectively. The method includes obtaining concentration of one or more electrolytes and metabolic content in a dialysate fluid flowing into the dialyser and out of the dialyser through a first dialysate tube and a second dialysate tube, respectively. The first dialysate tube and the second dialysate tube are arranged to pass through a third sensor and a fourth sensor configured in the haemodialysis filtering device, respectively. Further, the method includes identifying variations in the concentration obtained for the one or more electrolytes and the metabolic content in the blood sample with respect to the concentration obtained for one or more electrolytes and metabolic content in the dialysate fluid, respectively. Thereafter, the method includes performing removal of the one or more electrolytes and the metabolic content from the blood based on the identified variations.

In an embodiment, the present disclosure may relate to a haemodialysis filtering device for providing personalised haemodialysis for a subject. The haemodialysis filtering device comprises a first sensor configured to measure variations in the concentration of the one or more electrolytes in blood sample, a second sensor configured to measure variations in the concentration of the metabolic content in the blood sample. Further, the haemodialysis filtering device comprises a third sensor configured to measure variations in the concentration of the one or more electrolytes in dialysate fluid and a fourth sensor configured to measure variations in the concentration of the metabolic content in the dialysate fluid. The haemodialysis filtering device comprises a computing unit for identifying variations in the concentration obtained for the one or more electrolytes and the metabolic content in the blood sample with respect to the concentration obtained for one or more electrolytes and metabolic content in the dialysate fluid, respectively. Based on the identified variations, removal of the one or more electrolytes and the metabolic content from the blood sample is performed.

In an embodiment, the present disclosure may relate to a dialysis apparatus for providing personalised dialysis to subject. The dialysis apparatus includes a dialysing sensing device comprising a support structure in which the subject is laid down during dialysis, an Electrocardiography (ECG) acquisition unit connected to the subject through one or more ECG electrodes for acquiring ECG signals of the subject, a Blood Pressure (BP) and Ballistocardiography (BCG) signal acquisition unit connected to the subject for monitoring the BP of the subject, a Photoplethysmography (PPG) signal acquisition unit connected to a PPG sensor attached to the subject for monitoring urea in blood of the subject and a weight acquisition unit comprising one or more sensors connected to the subject for monitoring weight of the subject. Further, the dialysis apparatus includes an extracorporeal blood circuitry connected to the subject for drawing blood sample from the subject for haemodialysis and feeding back to the subject, a dialysis fluid circuitry configured for preparing dialysate solution, a dialyser connected to the dialysis fluid circuitry for receiving the dialysate solution and the blood sample from the extracorporeal blood circuitry, an optical spectral electrolyte estimation device for estimating the concentration of one or more electrolytes, a decision and control device configured for receiving inputs from the ECG acquisition unit, the BP and BCG signal acquisition unit, the PPG signal acquisition unit and the weight acquisition unit to assist in dialysis and a haemodialysis filtering device for providing personalised haemodialysis for a subject.

The foregoing summary is illustrative only and is not intended to be in any way limiting. In addition to the illustrative aspects, embodiments, and features described above, further aspects, embodiments, and features will become apparent by reference to the drawings and the following detailed description. For a better understanding of exemplary embodiments of the present invention, together with other and further features and advantages thereof, reference is made to the following description, taken in conjunction with the accompanying drawings.

### BRIEF DESCRIPTION OF THE DRAWINGS

The embodiments of the disclosure itself, as well as a preferred mode of use, further objectives, and advantages thereof, will best be understood by reference to the following detailed description of an illustrative embodiment when read in conjunction with the accompanying drawings. One or more embodiments are now described, by way of example only, with reference to the accompanying drawings in which:

FIG. 1 illustrates an exemplary environment for determining personalised haemodialysis for a subject in accordance with some embodiments of the present disclosure;

FIG. 2A illustrates an exemplary haemodialysis apparatus for providing personalised haemodialysis to subject in accordance with some embodiments of the present disclosure;

FIG. 2B shows an exemplary representation of dialysis sensing device in accordance with some embodiments of the present disclosure;

FIG. 2C illustrates an extracorporeal blood fluid circuit with various sensing systems in accordance with some embodiments of the present disclosure;

FIG. 2D illustrate an exemplary dialysis fluid circuit system with various sensing system for haemodialysis in accordance with some embodiments of the present disclosure;

FIG. 2E illustrate an exemplary decision and control system in accordance with some embodiments of the present disclosure;

FIG. 3A illustrates an exemplary online decision system for electrolytes imbalance in accordance with some embodiments of the present disclosure;

FIG. 3B and FIG. 3C illustrate online sensing, decision, and control systems for hemofiltration in accordance with some embodiments of the present disclosure;

FIG. 4A and FIG. 4B illustrates exemplary embodiments of monitoring metabolic waste products in blood fluid and dialysate fluid in accordance with some embodiments of the present disclosure;

FIG. 4C illustrates an exemplary optical setup for PPG based uraemia detection in accordance with some embodiments of the present disclosure;

FIG. 4D illustrates an exemplary embodiment of body weight measurement using in accordance with some embodiments of the present disclosure;

FIG. 5A illustrates an exemplary embodiment for monitoring electrolytes in blood fluid in accordance with some embodiments of the present disclosure;

FIG. 5B illustrates an exemplary embodiment for monitoring electrolytes in dialysis fluid in accordance with some embodiments of the present disclosure;

FIG. 5C illustrates a flowchart for adequacy of haemodialysis based on concentrations in accordance with some embodiments of the present disclosure; and

FIG. 6 illustrates a flowchart showing a method for determining personalised haemodialysis for a subject in accordance with some embodiments of the present disclosure.

### DESCRIPTION OF THE DISCLOSURE

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

Embodiments of the present disclosure relate to a method and a haemodialysis filtering device for providing personalised haemodialysis for a subject. Currently in existing systems dialysis systems, though the patient is a part of the bloodline circuit of dialysis process, neither blood electrolytes variations nor its dependent parameters are utilized for monitoring, controlling and alarming system on any eventualities. Typically, dialysis control restricts to physical parameters like flow/pressure in both blood and dialysis path. Further, there is a large gap in conventional HD where onetime generic prescription is provided with fixed dosage, when compared to natural human kidney, which is capable of maintaining complete homeostasis using complex sensing and biofeedback system to respond for any changing condition. Also, currently in intra dialysis process, continuous removal of fluid from individual dialysis patient may lead to either myocardial infarctions or any other cardiac related fatal issues, which demands to have the balance in either electrolytes parameters or physical parameters in an extracorporeal blood arm of the dialysis. Additionally, this gives rise to problem of dry weight maintenance and requires an immediate alert system.

The present disclosure relates to a method and haemodialysis filtering device for determining personalised haemodialysis for a subject. Typically, concentration is obtained for one or more electrolytes and of metabolic content in a blood sample of a subject flowing into a dialyser and out of the dialyser. Similarly, concentration of one or more electrolytes and metabolic content in a dialysate fluid flowing into the dialyser and out of the dialyser is obtained. Variations in the concentration obtained for the one or more electrolytes and the metabolic content in the blood sample is identified with respect to the concentration obtained for one or more electrolytes and metabolic content in the dialysate fluid, respectively. Thereafter, based on the variations, removal of the one or more electrolytes and the metabolic content from the blood based is performed. Thus, the present disclosure provides individualized preparation of dialysis to maintain balance of electrolytes and metabolic waste product to arrive at effective and safe haemodialysis.

FIG. 1 illustrates an exemplary environment for determining personalised haemodialysis for a subject in accordance with some embodiments of the present disclosure.

FIG. 1 shows a haemodialysis filtering device 101. The haemodialysis filtering device 101 includes blood sample 102 of a subject passing into and out of a dialyser 105 through a first blood bypass tube 103₁ and a second blood bypass tube 103₂, respectively. In an embodiment, the dialyser 105 is an apparatus in which dialysis is carried out. The dialyser 105 consists of essentially one or more containers for liquids separated into compartments by membranes. The first blood bypass tube 103₁ and the second blood bypass tube 103₂ are arranged in a first sensor 107 and a second sensor 109 which are configured in the haemodialysis filtering device 101, respectively. In an embodiment, the first sensor 107 and a second sensor 109 are micro-optofluidic Raman spectroscope devices. Particularly, the first sensor 107 measures variations in the concentration of the one or more electrolytes in blood sample 102 and the second sensor 109 measures variations in the concentration of the metabolic content in the blood sample 102.

Further, the hemodialysis filtering device 101 includes a fifth sensor 111 for detecting hemolysis condition of the blood sample 102 for accessing blood clot in parallel fashion.

Similar to the sample blood, the hemodialysis filtering device 101 obtains concentration of one or more electrolytes and metabolic content in a dialysate fluid flowing into the dialyzer 105 and out of the dialyser 105 through a first dialysate tube 113₁ and a second dialysate tube 113₂, respectively. The first dialysate tube 113₁ and the second dialysate tube 113₂ are arranged to pass through a third sensor 115 and a fourth sensor 117 configured in the hemodialysis filtering device 101, respectively. In an embodiment, the third sensor 115 and a fourth sensor 117 are micro-optofluidic Raman spectroscope devices. Particularly, the third sensor 115 measures variations in the concentration of the one or more electrolytes in dialysate fluid and the fourth sensor 117 measures variations in the concentration of the metabolic content in the dialysate fluid. Then, the hemodialysis filtering device 101 identifies by using a computing unit 119 variations in the concentration obtained for the one or more electrolytes and the metabolic content in the blood sample 102 with respect to the concentration obtained for one or more electrolytes and metabolic content in the dialysate fluid, respectively. Additionally, the micro-optofluidic devices may be arranged in sequence such that bypass blood paths are arranged in single tubing. In an embodiment, both in above cases, the blood used to evaluate any concentration is not used for feeding again to extracorporeal main blood path.

In an embodiment, when a difference occurs in concentration of one or more electrolyte and metabolic content in the blood sample and dialysis fluid, the hemodialysis filtering device 101 may facilitate to remove undesired electrolytes and metabolic content in blood by increasing efficiency in dialysis process. This may be true even when the electrolytes in blood are maintained at the personal prescription level of the subject under dialysis.

The concentration differences are shown in below equations. For instance, on denoting,

Δ_{B}=C_{B}_{ᵢ}−C_{B}_{ₒ} (1)

Δ_{D}=C_{D}_{ₒ}−C_{D}_{ᵢ} (2)

Where C_{B}_{ᵢ} and C_{B}_{ₒ} represents concentrations measured at the input and output of the dialyzer 105 in blood fluid flow circuit. Generally, concentration of metabolic content is higher than measured concentration. Similarly, C_{D}_{ᵢ} and C_{D}_{ₒ} represents concentrations measured at the concentrations at the input and output of dialyzer 105 in the dialysis fluid flow circuit. But, in dialysate flow circuit, the concentration of the output is higher than input side.

Further, denoting Q_{B} and Q_{D} as volume flow rates of blood and dialysate fluids, respectively.

In fact, in haemodialysis, the ratio between dialysate fluid volume to blood fluid is given by as

$$\frac{{Q}_{D}}{{Q}_{B}}$$

which is typically maintained as K=2, but must be greater than 1.

Ideally, at the end of dialysis process, mass transfer may be efficient, if the equation (3) is true:

Q_{B}Δ_{B}=Q_{D}Δ_{D} (3)

In practice, while achieving above goal over the period of the dialysis, it is required to target to reduce the factor mentioned below:

Mᵢ=Q_{B}Δ_{B}−Q_{D}Δ_{D} (4)

On setting, Q_{D}=k Q_{B}, the above target reduction process is viewed as:

Mᵢ=Q_{B}(kΔ_{B}=Δ_{D}) (5)

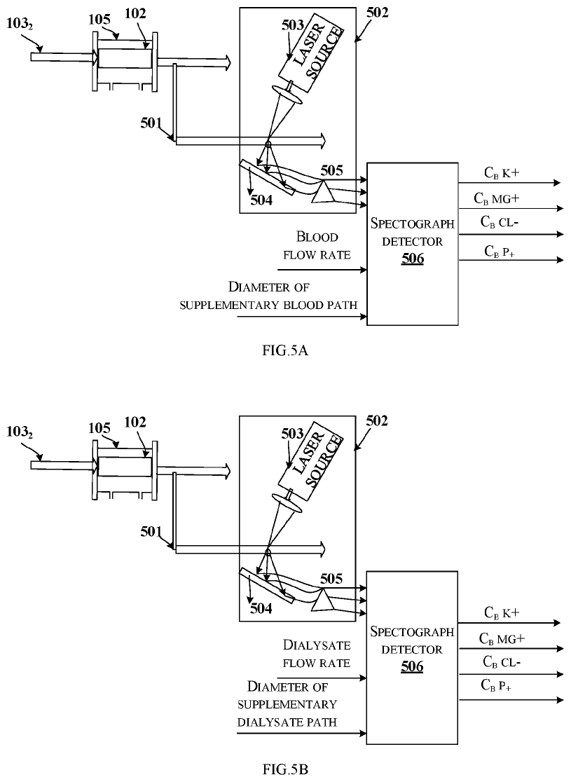

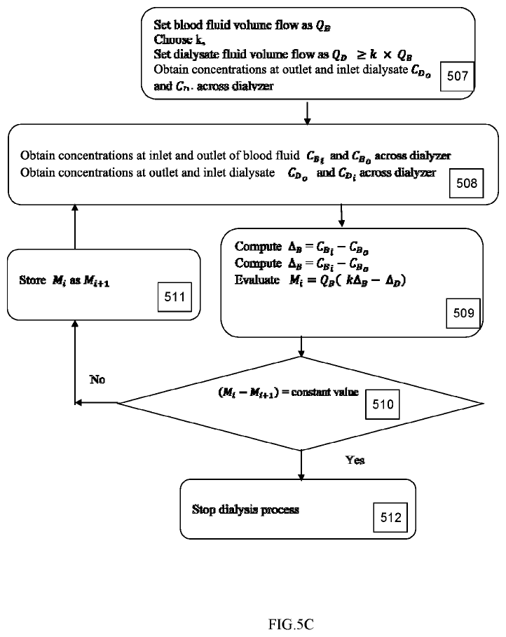

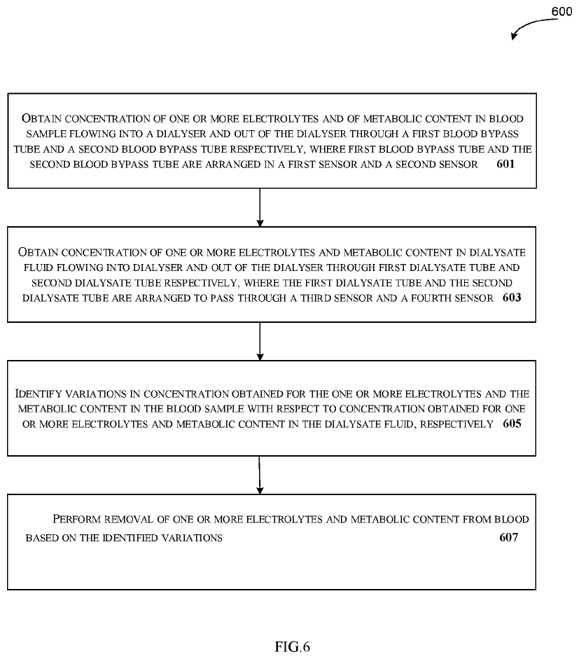

Based on the above, i^{th} instant to (i+1)th instant concentration change, (Mᵢ−Mᵢ₊₁), and the metabolic content/waste may progressively approach to a constant value, which may lead to an adequacy of haemodialysis. FIG. 5C illustrates a flowchart for adequacy of haemodialysis based on concentrations in accordance with some embodiments of the present disclosure. FIG. 5G describes a process of identifying adequacy of dialysis using several concentration measurements and keeping the flow rate of blow rates of blood and dialysate fluid rate.

At block 507, the blood fluid rate and the dialysate fluid rate are set as definite proportion to blood fluid rate.

At block 508, the concentrations of any metabolic wastes are obtained at both at the beginning and end of dialyzer 105 for the blood as well as for the dialysate fluid. Further, differences between them are identified as mentioned in block 509. Also, at block 509, a target factor Mᵢ=Q_{B}(kΔ_{B}−Δ_{D}) is evaluated.

Further, at block 510, the above target factors are compared with previous instant target factor. When the target factors are greater, at block 511, the dialysis is continued by storing the current target factor. However, upon meeting the target, at block 512, the decision and control factor is indicated to stop the dialysis process, since it reaches the adequacy.

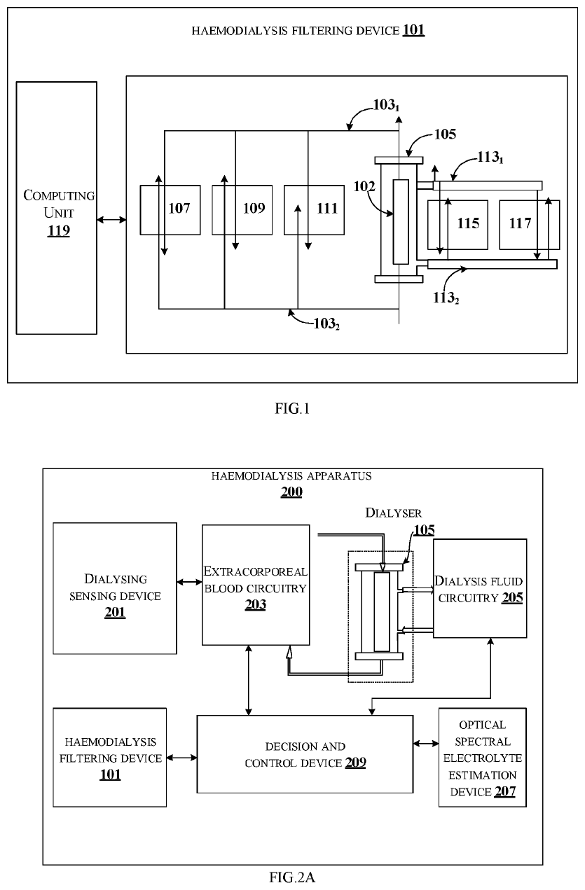

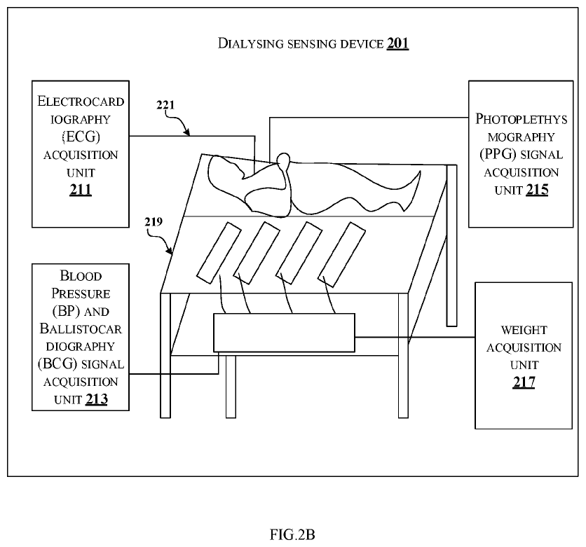

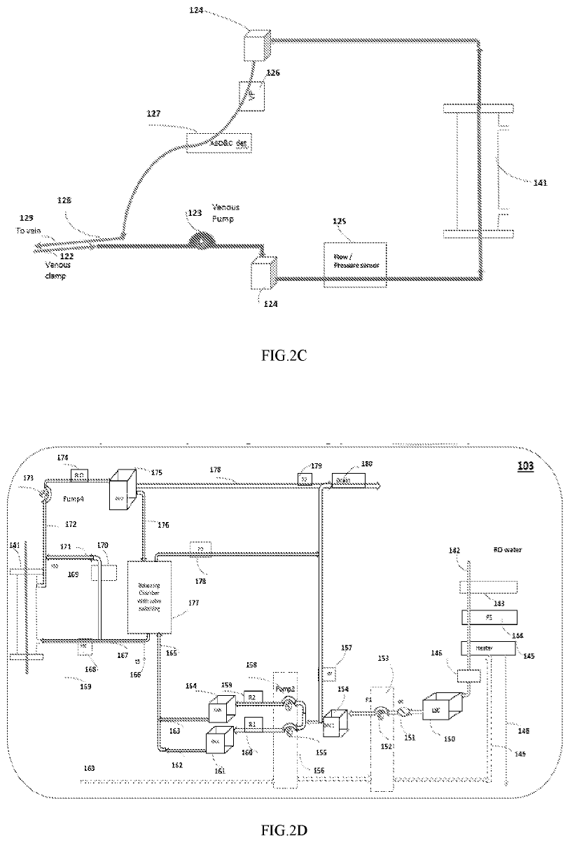

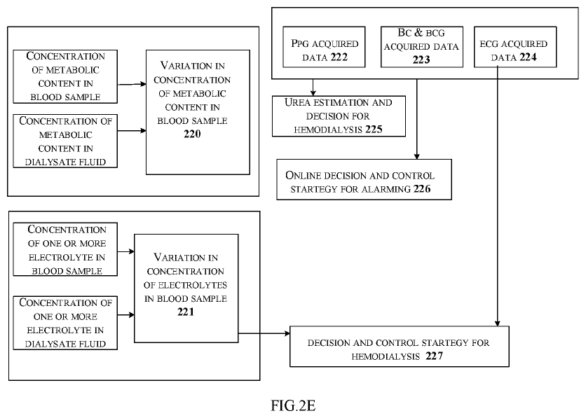

FIG. 2A illustrates an exemplary haemodialysis apparatus for providing personalised haemodialysis to subject in accordance with some embodiments of the present disclosure.

FIG. 2A shows a haemodialysis apparatus 200 for providing personalised dialysis to the subject. The haemodialysis apparatus 200 comprises a dialysing sensing device 201, an extracorporeal blood circuitry 203, the dialyser 105, a dialysis fluid circuitry 205, an optical spectral electrolyte estimation device 207, a decision and control device 209 and the haemodialysis filtering device 101. FIG. 2B shows an exemplary representation of dialysis sensing device in accordance with some embodiments of the present disclosure. As shown, FIG. 2B comprises a patient 221 lying on a support structure 219 which is arranged to draw blood that requires removal of several metabolic waste products, toxins, etc., and a balancing of multiple electrolytes. The dialyzing sensing device 201 comprises an ECG acquisition device 214 which facilitates to acquire ECG signal of the patient 221 through set leads depending upon number of ECG electrode connected (for example, 12 leads are preferred but figure depicts five electrodes symbolically). Additionally, the dialyzing sensing device 201 includes a BP and BCG acquisition system 213 The support structure 219 is supported with attached electronic weigh scale sensor and device and acquired through lead and a weight acquisition unit 217. Further, dialyzing sensing device 201 includes a PPG signal acquisition system 215 which facilitates to obtain signals from the patient 221 through a lead. FIG. 2C illustrates an extracorporeal blood fluid circuit with various sensing systems in accordance with some embodiments of the present disclosure;

As shown, in FIG. 2C, blood from the patient is drawn from vein using blood access device (referred to as fistula) 122 with venous clamp by using blood pump 123 and is fed to a venous drip chamber 124 that collects adequate amount of blood. Further, the blood flow or pressure is measured using flow/pressure sensor 125 with inlet to the dialyzer 141 with specified flow rate. A blood outlet of the dialyzer 141 is collected at another drip chamber 124. Further, the blood is fed to vein using feeding fistula 129 after monitoring air bubble in blood using any suitable air bubble detector depending on decision on allowable limits amount of air bubbles. Moreover, the blood is fed through 129 to vein if the blood is not clot, wherein the blood clot detection is performed by haemolysis detector. Simultaneously, the detection may also be dependent on blood flow or pressure flow.

FIG. 2D illustrate an exemplary dialysis fluid circuit system with various sensing system for haemodialysis in accordance with some embodiments of the present disclosure. As shown, FIG. 2D describes the dialysis fluid circuit system having various sensing system 103. The dialysis fluid circuitry 205 comprising various components and sensors. A reverse osmosis (RO) water source 142 that is needed for dialysis fluid generation is checked with initial temperature and flow rate using temperature sensor 143 and flow sensor 144 respectively before heating by a heater 145. Further, the RO water is heated to a predefined temperature using heater 145 to assure the dialysis fluid is maintained in the predefined temperature and measured using another temperature sensor 146. In order to regulate the temperature of dialysis fluid, the monitored temperature parameters are supplied to the decision and control unit. Also, a control path 149 sends temperature data to decision and control device 209 wherein the decision and control device 209 responds back with required information to maintain the predefined temperature for maintaining the RO water warmness.

Further, the water is made to flown into a de-aeration chamber 154 using degassing pump 152 via orifice 151. The De-aerated RO water is pumped into a de-aeration acid chamber 159 and de-aeration bicarbonate chamber 161 using pumps 155 and 158, whose pump speed is controlled and regulated by decision and control system device 209. An outlet from 159 and 161 from the two de-aeration chambers is mixed to form common dialysis fluid path 155 and the same serve as one of the inlet 155 to the balancing chamber 165.

Typically, a balancing chamber contains two chambers separated by thin diaphragm having an outlet 177 which contain two sets of inlets and outlets. Also, another outlet path 166 is serving as inlet to dialyzer 141. An inlet path 176 to the balancing chamber is served from the de-aeration chamber 175. An outlet of the dialyzer fluid is pumped out using suction pump 173 via outlet path 172 and is checked for any blood leakage using a blood leak detector 174 before colleting in de-aeration tank 175. Another outlet 178 from de-aeration tank is facilitated to drain tank 180. Further, a bypass path 171 is provided which includes a pressure sensor 170, which is used to prime or rinse the dialyser before and after the dialysis process. In an embodiment, the dialysis fluid temperature is measured using temperature sensor 166 to check and assure the temperature of dialysis fluid at a predefined level. Further, the dialysis fluid circuitry 205 includes an air bubble detector 128 to monitor presence of number of air bubbles and air quality. Also, arterial pressure is sensed using sensors to monitor and regulate the blood supply pressure to the body before feeding.

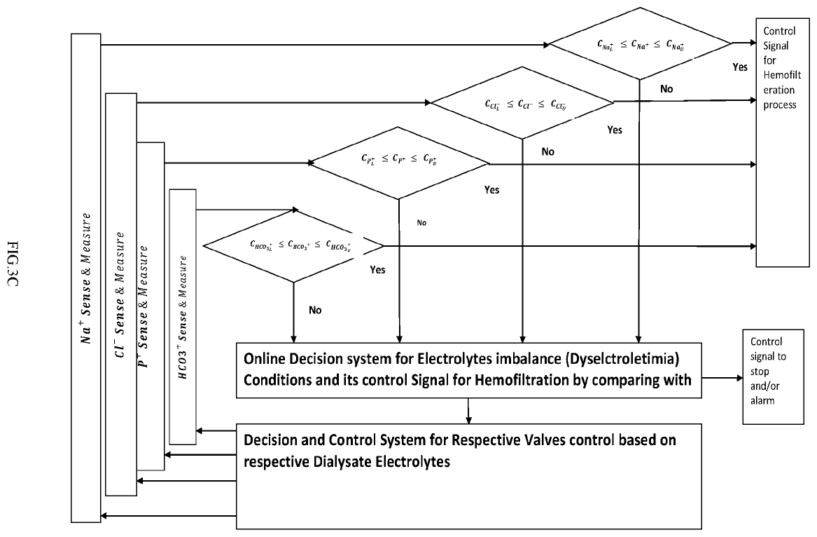

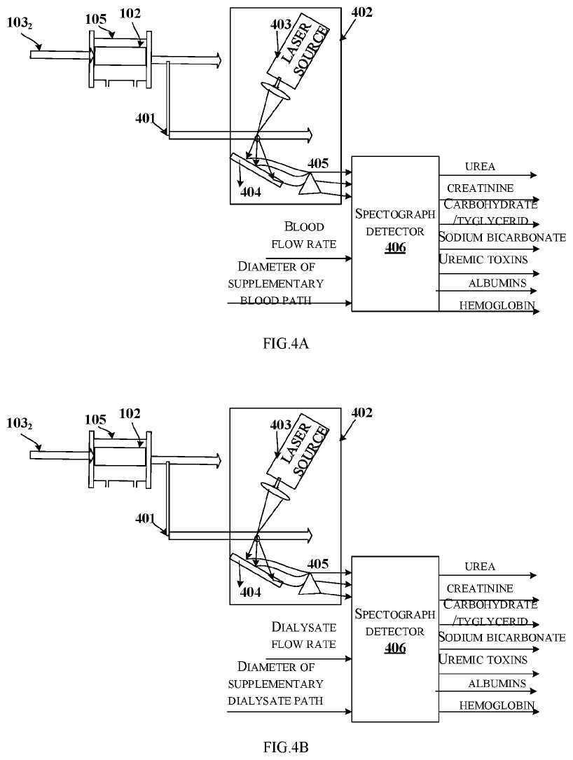

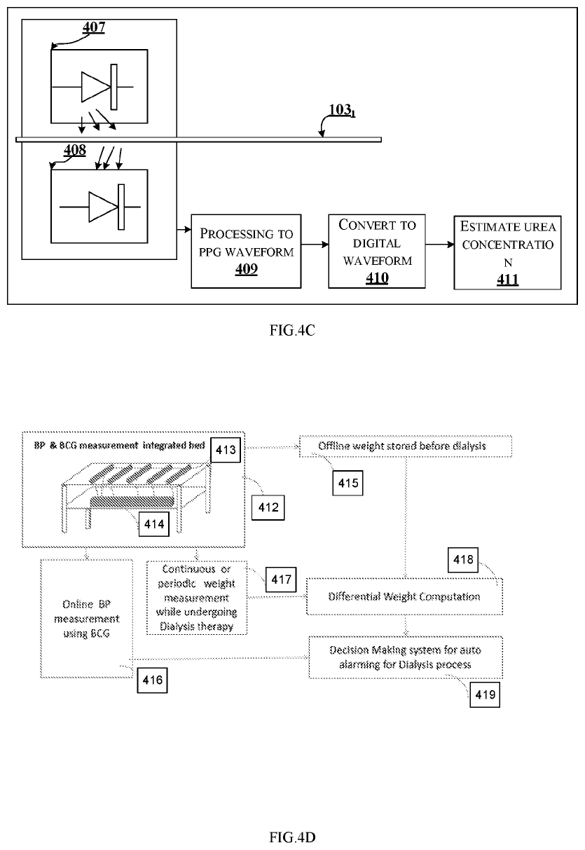

FIG. 2E illustrate an exemplary decision and control system in accordance with some embodiments of the present disclosure. FIG. 2E may include embodiments shown in FIG. 4A and FIG. 4B for identifying concentration of multiple metabolic waste products in both blood path and dialysis path. The concentration of multiple metabolic waste products in both blood path and dialysis path is obtained by an optical spectral metabolic estimation device (207) as shown in FIG. 2A. Similarly, the decision and control device 209 may include embodiments of FIG. 5A and FIG. 5B for evaluating differential multiple electrolytes concentration values between blood fluid and dialysis fluid. As shown in FIG. 2E, an online decision of auto alerting 226 is raised based on differential value of one or more metabolic waste products limits or dyselectroletimea condition, which is dependent on online ECG 224, PPG 222 and evaluated concentration of urea estimation value limit 225. Additionally, the alert signal is also taken as per online BP and BCG data 223. Further, at 227, the decision and control for haemodialysis is provided to evaluate appropriate mixing. In an embodiment, the decision may be based on allowable condition of one or more electrolytes which is decided using both qualitative ECG signal 220 and quantitative affirmation form values as per block 221.

FIG. 4C illustrates an exemplary optical setup for PPG based uraemia detection in accordance with some embodiments of the present disclosure. FIG. 4C illustrates an exemplary optical setup for PPG based uraemia detection in accordance with some embodiments of the present disclosure. FIG. 4C describes an optical setup for PG and uraemia detection. The setup includes generation of analog PPG signal based on arrangement shown in FIG. 4C. The arrangement may involve NIR light rays generated by photodiode 407 to fall on blood tube 103₁ arranged on a finger of the patient. Further, the setup includes a photodiode detector 408, which is positioned on the opposite side of the blood tube 103₁. An analog signal is appropriately conditioned at block 409. At block 410, the PPG signal is converted to digital PPG signal. This signal is utilized to find the uremic concentration for decision and control at 411.

FIG. 4D. illustrates an exemplary embodiment of body weight measurement using in accordance with some embodiments of the present disclosure. As shown in FIG. 4D, a BP and BCG acquisition system are shown at block 412, which is used for acquisition of BP and BCG at block 416. At the same time at block 417, the system collects continuous or periodic weight using attached weight scale sensor 414 and electrode 413. At block 418, a differential weight is calculated using reference weight as per block 415. At block 419, measured data is provided to the decision control system for auto alarming for either continuation or stop of dialysis process.

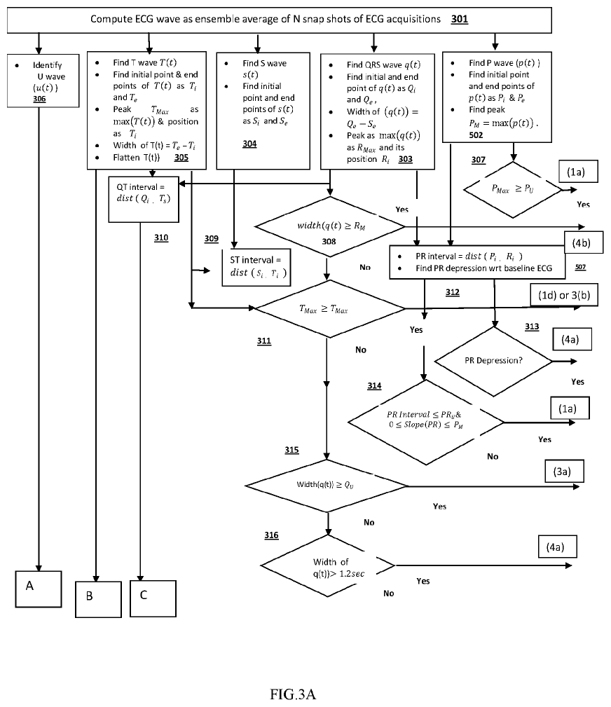

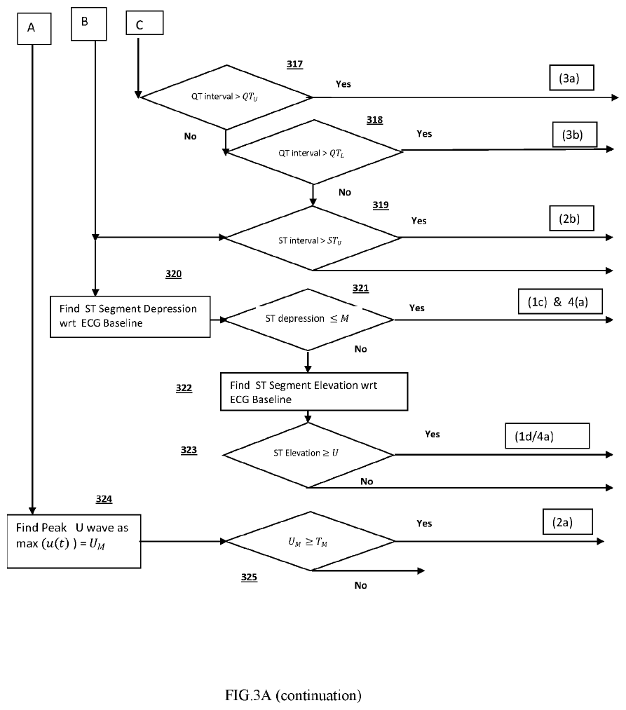

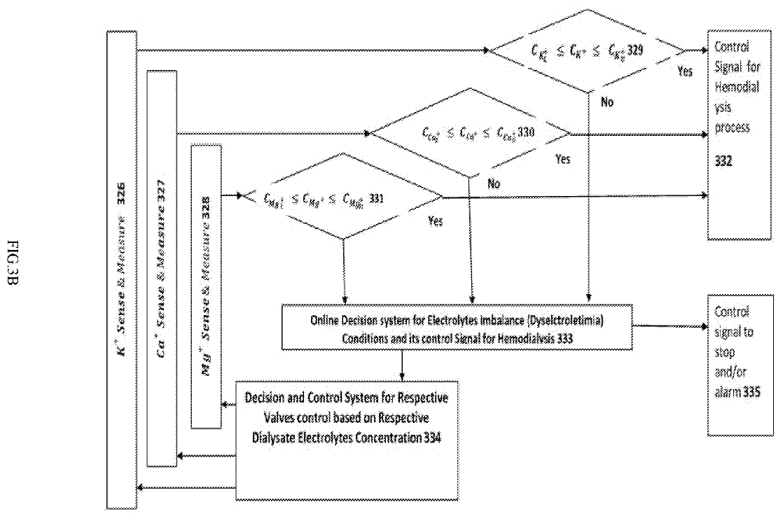

FIG. 3A illustrates an exemplary online decision system for electrolytes imbalance in accordance with some embodiments of the present disclosure. FIG. 3A discloses an online decision system for electrolytes imbalance (Dyselectroletimea) condition and generation of control signal for haemodialysis using ECG signal acquired either continuously or periodically using ECG acquisition equipment.

At bock 301, an ECG wave is calculated as an ensemble average of “n” snap shots of ECG acquisitions, where “n” is preferred to be more than ten.

At 302, considering the ECG obtained at block 301, P wave, p(t) is computed, and finding initial point and end points of p(t) as Pᵢ&Pₑ and peak P_{M}=max(p(t)).

At block 303, calculated QRS wave q(t), initial and end point of q(t) as Qᵢ and Qₑ. Further, width of wave q(t)=Qₑ−Sₑ and Peak as max(q(t)) as R_{Max}, and its position Rᵢ is computed.

At block 304, S wave s(t) and initial point and end points of s(t) as Sᵢ is computed.

At block 305, T wave T(t), initial point & end points of T(t) as Tᵢ and Tₑ and Peak T_{Max} as max(T(t))& position as Tᵢ is identified. Further, width of T(t)=Tₑ−Tᵢ is computed.

At block 306, U wave (u(t) is identified.

At block 307, check P_{Max}≥P_{U}, then (1a) (Pericarditis).

At block 308, check width(q(t)≥R_{M}, if yes decide (4b-Hyper-magnesia).

At block 309, ST interval=dist is computed. At block 310, QT interval=distancebetween(Qᵢ·Tₛ) is computed. At block 311, check if T_{Max}≥T_{M}, where T_{M} is pre-decided. If yes, then true then 1(d) (Ischemia/Hypomagnesemia) or 3(b)(Hypercalcemia) is decided.

At block 312, compute PR interval as distance between(Pᵢ·Rᵢ). At block 313, check if PR is depressed lower than the pre-decided value. If yes, then decides (4a) (Hypomagnesemia).

At block 314, check both PRInterval≤PR_{U} and 0≤Slope(PR)≤P_{M}. If yes, then decide (1a) (Pericarditis).

At block 315, check Width of (q(t))≥Q_{U}. If yes, then decide (3a), otherwise move to block 316. At 316, check whether width of q(t))>1.2 sec. If yes, decide 4(a) (Hypomagnesemia).

At block 317, check if QT interval>QT_{U}, where QT_{U} is a predefined value. If yes, decide (3a) (Hypo-calcimia). Otherwise, move to block 318. At block 318, check if QT interval>QT_{L}. If yes, decide (3b) (Hypercalcemia), otherwise move to block 319.

At block 319, check if ST interval>ST_{U}, where ST_{U} is predefined upper value. If yes, decide (2a) (Hypokalaemia).

At block 320, check if ST segment is depressed with reference to baseline ECG.

At block 321, check if ST depression is lesser than value M. If true, decide (1c) (Acute MI) and (4a) (Hypomagnesemia). Otherwise, check ST segment with respect to baseline ECG at block 322.

At block 323, check if ST elevation is greater than U, where U is a predetermined value. If yes, decide (1d/4a) (Ischemia/Hypomagnesemia)/(Hypomagnesemia) otherwise.

At block 324, identify peak of U wave as set as U_{M}. If true, move to block 325.

At block 325, check whether U_{M}≥T_{M}. If true, decide (2a) (Hypokalaemia).

FIG. 3B and FIG. 3C illustrate online sensing, decision, and control systems for hemofiltration in accordance with some embodiments of the present disclosure.

FIG. 3B describes an online sensing, decision, and control system for Haemodialysis. At block 326, 327 and 328, potassium, calcium, magnesium electrolyte concentration is sensed and measured, respectively. Further, electrolyte concentration is checked respectively at blocks 329, 330 and 331 to find whether it in between higher range through lower range values. In case, the electrolyte is in higher range, method invokes a control signal to haemodialysis is as described in block 332. Alternatively, an online decision is performed for electrolytes imbalance at bock 333. At block 334, a decision is performed for respective valves control based on respective dialysate electrolytes concentration. At block 335, control signal is stopped for alarm.

Similarly, embodiment shown in FIG. 3C for sodium, phosphate, chlorine, and bicarbonate concentrations is evaluated quantitatively only by optical method.

FIG. 5A illustrates an exemplary embodiment for monitoring electrolytes in blood fluid in accordance with some embodiments of the present disclosure. Similarly, FIG. 5B illustrates monitoring of multiple electrolytes concentration quantitatively using both inlet dialysis fluid and outlet dialysis fluid 501 fed by an auxiliary path, which possess same flow rate and scaled down supplementary tubing. At block 502, a Near Infrared (NIR) laser source generates a laser induced breakdown which is collected through a prism 505 to give rise to a Raman spectrum. At block 506, concentration dialysis fluid for potassium, Magnesium, phosphate, and chlorides are obtained.

FIG. 6 illustrates a flowchart showing a method for determining personalised haemodialysis for a subject in accordance with some embodiments of the present disclosure.

As illustrated in FIG. 6, the method 600 includes one or more blocks for determining personalised haemodialysis for a subject. The method 600 may be described in the general context of computer executable instructions. Generally, computer executable instructions can include routines, programs, objects, components, data structures, procedures, modules, and functions, which perform particular functions or implement particular abstract data types.

The order in which the method 600 is described is not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

At block 601, obtaining concentration of one or more electrolytes and of metabolic content in the blood sample 102 of the subject flowing into the dialyser 105 and out of the dialyser 105 through the first blood bypass tube 103₁ and the second blood bypass tube 103₂, respectively. The first blood bypass tube 103₁ and the second blood bypass tube 103₂ are arranged in the first sensor 107 and the second sensor 109 configured in the haemodialysis filtering device 101, respectively.

At block 603, obtaining concentration of one or more electrolytes and metabolic content in the dialysate fluid flowing into the dialyser 105 and out of the dialyser 105 through the first dialysate tube 113₁ and the second dialysate tube 113₂, respectively. The first dialysate tube 113₁ and the second dialysate tube 113₂ are arranged to pass through the third sensor 115 and the fourth sensor 117 configured in the haemodialysis filtering device 101, respectively.

At block 605, identifying variations in the concentration obtained for the one or more electrolytes and the metabolic content in the blood sample 102 with respect to the concentration obtained for one or more electrolytes and metabolic content in the dialysate fluid, respectively.

At block 607, performing removal of the one or more electrolytes and the metabolic content from the blood based on the identified variations.

Advantages of the Present Disclosure

An embodiment of the present disclosure provides online differential value instead of absolute value to make personalized or individual prescription continuously.

An embodiment of the present disclosure provides individualized preparation of dialysis to maintain balance of electrolytes and metabolic waste product to arrive at effective and safe haemodialysis.

An embodiment of the present disclosure provides personalized adequacy of haemodialysis which keeps safety of the patient.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 6 show certain events occurring in a certain order. In alternative embodiments, certain operations may be performed in a different order, modified, or removed. Moreover, steps may be added to the above-described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of providing personalised haemodialysis for a subject, the method comprising:
obtaining, by a haemodialysis filtering device, concentration of one or more electrolytes and of metabolic content in a blood sample of a subject flowing into a dialyser and out of the dialyser through a first blood bypass tube and a second blood bypass tube respectively, wherein the first blood bypass tube and the second blood bypass tube are arranged in a first sensor and a second sensor configured in the haemodialysis filtering device respectively;
obtaining, by the haemodialysis filtering device, concentration of one or more electrolytes and metabolic content in a dialysate fluid flowing into the dialyser and out of the dialyser through a first dialysate tube and a second dialysate tube respectively, wherein the first dialysate tube and the second dialysate tube are arranged to pass through a third sensor and a fourth sensor configured in the haemodialysis filtering device respectively;
identifying, by the haemodialysis filtering device, variations in the concentration obtained for the one or more electrolytes and the metabolic content in the blood sample with respect to the concentration obtained for one or more electrolytes and metabolic content in the dialysate fluid, respectively; and
performing, by the haemodialysis filtering device, removal of the one or more electrolytes and the metabolic content from the blood based on the identified variations.

2. The method as claimed in claim 1, wherein the first sensor measures variations in the concentration of the one or more electrolytes in blood sample and the second sensor measures variations in the concentration of the metabolic content in the blood sample.

3. The method as claimed in claim 1, wherein the third sensor measures variations in the concentration of the one or more electrolytes in dialysate fluid and the fourth sensor measures variations in the concentration of the metabolic content in the dialysate fluid.

4. The method as claimed in claim 1, wherein detecting by a fifth sensor configured in the hemodialysis filtering device, hemolysis condition of the blood sample flowing through a third blood bypass tube into the dialyzer.

5. The method as claimed in claim 1, wherein obtaining the concentration of the metabolic content in the blood fluid and the dialysate fluid comprises:
causing a laser light source to excite light beams on the blood sample and the dialysate fluid drawn from the first blood bypass tube and the first dialysate tube respectively;
providing optical signals by a fibre optics tube based on the light beams;
detecting by a prism one or more spectrums based on the optical signals; and
obtaining by a spectrograph detector concentration of metabolic content in the blood sample and the dialysate fluid based on the one or more spectrums.

6. The method as claimed in claim 1, wherein estimating concentration of uremia in the blood sample by:
emitting light rays to a blood flow tube arranged between a Near Infrared (NIR) LED source and a photodiode detector;
converting the light rays projected from the NIR LED source as digital PPG signals; and
estimating the uraemia concentration in the blood sample based on the PPG signals.

7. The method as claimed in claim 1, wherein measuring body weight by:
acquiring BP and BCG signals by signal acquisition unit;
collecting one of continuous or periodic weight using a weight acquisition unit, wherein variation in weight of the subject is calculated between the collected weight with a predefined reference weight;
providing the acquired BP signal, BCG signal, and the variation in weigh to a decision and control device for taking one or more actions for providing personalized hemodialysis to the subject.

8. The method as claimed in claim 7, wherein the one or more actions comprises generating alarms for either continuation or stop of hemodialysis for the subject.

9. The method as claimed in claim 1, wherein removal of the one or more electrolytes and the metabolic content comprises controlling respective valves based on the variations.

10. The method as claimed in claim 1, wherein computing initial and end point of a plurality of waves of an ECG signal for checking electrolytes imbalance condition and generating one or more control signals for hemodialysis.

11. The method as claimed in claim 1, wherein providing personalized hemodialysis for the subject further comprising identifying adequacy of hemodialysis by:
setting blood fluid rate and dialysate fluid rate as predefined proportion to blood fluid rate;
obtaining variations in the concentration obtained for the metabolic content in the blood sample with respect to the concentration obtained for the metabolic content in the dialysate fluid;
evaluating a target factor based on ratio between volume of the dialysate fluid to the blood sample;
comparing the target factor with a previous target factor, wherein performing one of:
storing the target factor and continuing the haemodialysis when the target factor is greater than the previous target factor; or
indicating to stop the haemodialysis when the target factor is lesser or equal to the previous target factor.

12. A haemodialysis filtering device for providing personalised haemodialysis for a subject, comprising:
a first sensor configured to measure variations in the concentration of the one or more electrolytes in blood sample;
a second sensor configured to measure variations in the concentration of the metabolic content in the blood sample;
a third sensor configured to measure variations in the concentration of the one or more electrolytes in dialysate fluid;
a fourth sensor configured to measure variations in the concentration of the metabolic content in the dialysate fluid; and
a computing unit for identifying variations in the concentration obtained for the one or more electrolytes and the metabolic content in the blood sample with respect to the concentration obtained for one or more electrolytes and metabolic content in the dialysate fluid,
respectively, wherein removal of the one or more electrolytes and the metabolic content from the blood sample is performed based on the identified variations.

13. The hemodialysis filtering device as claimed in claim 12, wherein a fifth sensor configured for detecting hemolysis condition of the blood sample flowing through a third blood bypass tube into the dialyzer.

14. A hemodialysis apparatus for providing personalized hemodialysis to subject, the hemodialysis apparatus comprises:
a dialysing sensing device comprising:
a support structure in which the subject is laid down during haemodialysis;
an Electrocardiography (ECG) acquisition unit connected to the subject through one or more ECG electrodes for acquiring ECG signals of the subject;
a Blood Pressure (BP) and Ballistocardiography (BCG) signal acquisition unit connected to the subject for monitoring the BP of the subject;
a Photoplethysmography (PPG) signal acquisition unit connected to a PPG sensor attached to the subject for monitoring urea in blood of the subject; and
a weight acquisition unit comprising one or more sensors connected to the subject for monitoring weight of the subject;
an extracorporeal blood circuitry connected to the subject for drawing blood sample from the subject for haemodialysis and pushing back to the subject;
a dialysis fluid circuitry configured for preparing dialysate solution;
a dialyser connected to the dialysis fluid circuitry for receiving the dialysate solution and the blood sample from the extracorporeal blood circuitry;
an optical spectral metabolic estimation device for estimating concentration of one or more metabolic content in the blood sample and dialysate fluid;
a decision and control device configured for receiving inputs from the ECG acquisition unit, the BP and BCG signal acquisition unit, the PPG signal acquisition unit, and the weight acquisition unit to assist in dialysis; and
a haemodialysis filtering device for providing personalised haemodialysis to the subject.
